(19) United States
(12) Patent Application Publication
(10) Pub. No.: US 2007/0227543 A1
(43) Pub. Date: Oct. 4, 2007

(54) SUCTION CATHETER FOR ENDOTUBE AND METHODS OF MANUFACTURE AND OPERATION THEREOF

(75) Inventor: Cory W. Peichel, Richardson, TX (US)

Correspondence Address: HITT GAINES P.C., P.O. BOX 832570, RICHARDSON, TX, 75083, US

(73) Assignee: Cory Peichel

(21) Appl. No.: 11/278,468

(22) Filed: Apr. 3, 2006

(51) Int. Cl. A61M 31/00; A62B 9/06

(52) U.S. Cl. 128/207.14; 604/93.01

(57) ABSTRACT

A suction catheter and a method of manufacturing the same. In one embodiment, the suction catheter includes: (1) a housing portion having an interior passage therethrough, the interior passage couplable to a vacuum source, (2) a tube portion coupled to the housing portion protruding from the housing a length based on a length of an endotube to be suctioned and (3) a bypass valve associated with the housing portion and configured to control suction through the tube portion.

### CROSS-REFERENCE TO RELATED APPLICATION

This application is based on, and claims priority of, U.S. Provisional Patent Application Ser. No. 60/714,986, filed by Peichel on Sep. 8, 2005, entitled “Easy-To-Use, Clean-Store Suction Catheter,” commonly owned herewith and incorporated herein by reference.

### TECHNICAL FIELD OF THE INVENTION

The present invention is directed in general to suction catheter assemblies and, more particularly, to assemblies that can be used to aspirate secretions from tracheostomy tubes and endotracheal tubes and then stored in an sheath and methods of manufacturing and operating the same.

### BACKGROUND OF THE INVENTION

Rationale for the use of endotracheal and tracheostomy tubes, hereafter referred to generally as endotubes, varies. An endotracheal tube might be placed as a simple preoperative precaution, or a tracheostomy tube could be positioned for a lifetime of support to a damaged trachea. A tracheal tube is placed in a patient's respiratory tract where it creates an artificial airway to keep the natural trachea open so that the lungs can be properly ventilated. Because this artificial airway bypasses normal respiratory functions, the lower trachea and lungs produce more than usual amounts of mucus that must be removed.

Normally, mucus can be discharged from the respiratory tract by natural means such as by coughing. However, an intubated patient's ability to clear respiratory mucus is severely diminished, particularly if the patient is incapacitated. Therefore, the removal of secretions is typically accomplished by mechanical means with equipment that includes a vacuum source, connection tubing, a collection canister and a suction catheter.

The use of suction catheters to remove tracheobronchial secretions from intubated and tracheostomized patients is well documented. These catheters are made of a flexible medical grade material such as non-radiopaque vinyl, polyethylene, urethane or a soft polymer material. Open suction catheters, commonly used today, have elongated tubes sometimes enveloped by clear plastic sleeves, use fingertip-control valves to regulate flow and come with a variety of tips—some with side inlets to minimize trauma to delicate mucosal tissue.

A bypass valve allows flow at the tip of the catheter to be controlled. When the valve is open, air enters in through the aperture. When it is closed, air enters in through the tip of the catheter. To use the catheter the elongated tube of the device is advanced into and through the tracheal tube. Negative pressure is applied to one side of the collection canister, which draws a vacuum on the connection tubing and suction catheter. Because the bypass valve is open, air is drawn in through the uncovered aperture. With a finger covering the bypass valve, negative pressure is extended to the tip of the catheter, which pulls in mucus and other secretions from the patient's airway and from the interior of the tracheal tube.

Before the clinician inserts the suction catheter into the tracheal tube, she determines which mark on the catheter corresponds to the length of the tracheal tube and then monitors closely, taking care not to insert the catheter past that mark.

As Bell, et al., point out in U.S. Pat. No. 5,653,232, the length of most currently used catheters is typically between 20 and 24 inches. This length allows the catheters to be used for both nasopharyngeal and tracheobronchial suctioning. For normal, pre-measured suctioning, the catheter's excessive length permits the clinician to inadvertently introduce the catheter past the end of the tracheal tube and possibly damage the mucosa within the lower trachea and bronchial passages. Bell addressed this potential problem by allowing the length of the catheter to be decreased to match the length of the trachea. Bell addresses the potential for damage to the bronchial passages, but unfortunately, he did not address the potential for damage to the lower trachea.

Another problem encountered during the normal course of care is that the clinician must spend valuable time to find the predetermined depth mark each time the patient needs to be suctioned. Since care is administered 24 hours per day, finding that mark is sometimes difficult. Also because the catheter is excessively long, the clinician must use two hands to suction patients. If it is a sleeved catheter, she first pushes the wrapper back to the predetermined depth mark and holds it in place. As the clinician plunges the catheter into the tracheal tube with one hand holding the distal portion of the catheter, she closes the bypass valve with the thumb of the other. This can be a difficult and time-consuming procedure, especially in the middle of the night.

In its simplest form the bypass valve comprises an aperture in the wall of the catheter housing which is open to the atmosphere and controlled by a finger or a thumb. In other forms the valve is sealed to prevent cross contamination of infectious diseases. Simplicity of the valve drives cost, and for that reason the buying public tends to prefer devices with open apertures. This is true for open suction catheters used only once in a clinical environment and for devices used for an entire day in a home environment. Incremental benefits of the more complex valves do not seem to provide enough benefit to sway the purchasing decisions of users.

Valves of commonly used devices are similar to that described in U.S. Pat. No. 3,610,242. The '242 patent employs a split valve with one branch extending forward to the catheter tube, one branch extending forward to the finger-control aperture, and the trunk reaching back to the coupling that interfaces to the suction tubing. One problem with this approach is that the hand of the operator is drawn forward, and if there were an attempt to seat the catheter completely into the tracheal tube, a finger might touch and contaminate the inlet of the tracheal tube. A valve such as the one described in U.S. Pat. No. 3,375,828 ostensibly corrects for that problem because it portrays the aperture to the side of the catheter housing. Unfortunately, that type of valve suffers from an inability to isolate the operator's control finger from the suction fluid stream.

Suction catheters used in a clinical setting are normally packaged with latex gloves, used once and then discarded. Suction catheters used in a home environment often incorporate flexible, protective sleeves. While a substantial amount of mucus is withdrawn through the lumen of the suction catheter, a portion of those secretions remains as a film on the outside of the catheter. For a use-once catheter this is of no concern, but for a home-use catheter, which is used for an entire day, the secretions accumulate into quite a mess. Over the course of a day the catheter may be placed under a pillow, draped over a crib railing, slipped into an adjacent drawer, or tucked into the suction machine bag where it can and does fall to the floor. Mucus remaining on the catheter and sleeve become quite soiled in this unfriendly environment.

The clinician can use sterile or distilled water to clean the catheter after it has been used to suction a patient. She maintains her initial grip on the sleeve at the distal end of the catheter, plunges the catheter into a cup of water stored nearby and suctions the liquid up through the catheter and connection tubing. The water helps to loosen and remove secretions from the interior of the catheter. Unfortunately, mucus can still remain on much of the exterior of the catheter, which is often touched by an ungloved caregiver who must hold it near the edge of the sleeve. The sleeve is slid up and down the catheter tube with each use where it picks up the mucus from the catheter tube, contaminating the user and a good portion of the catheter tube.

In U.S. Pat. No. 4,898,586 McDonough addresses some of these problems with a stopper sealed sheath holder that protects the catheter if it accidentally slips to the floor. Because McDonough's rigid sheath is separate from the catheter, its internal surface is not exposed to outside contaminates in the same way that a fragile plastic sleeve might be. The first shortfall of this particular embodiment, though, is that it does not cover and protect the valve assembly and catheter housing. Second, this embodiment suffers from the expectation of an excessively long catheter. Once the catheter separates from its protective sheath, it needs to be gripped distally when used. Therefore, either the operator requires gloves or the catheter needs to be discarded after use, obviating the need for the protective sheath. Finally, this embodiment suffers from the required use of a costly stopper to seat the catheter into the sheath.

To summarize the above, all suction catheter assemblies heretofore known suffer from a number of disadvantages which include:

(a) The length of the catheter does not match the length of the tracheal tube. This permits a clinician to inadvertently introduce the catheter past the end of the tube and possibly damage either the mucosa within the lower trachea or the mucosa within bronchial passages.

(b) Because of the excessive length of today's suction catheters, the clinician must use two hands to operate the device, one to steer the tip of the catheter and one to manage the bypass valve.

(c) Because of the excessive length of today's suction catheters, the clinician must spend valuable time to align a pre-marked length with the opening of the tracheal tube to ensure that the tip of the catheter does not extend beyond the tip of the tube.

(d) Contamination of catheters used for an entire day is a serious concern. Mucus secretions often remain as a film on the outside of the catheter, and sleeves offer little protection, as the catheter is stored in a variety of locations that can damage and contaminate a very fragile sleeve.

Accordingly, what is needed in the art is a suction catheter that is relatively easy to use, is designed such that tissue damage is reduced and is capable of being stored in a relatively clean and convenient manner.

### SUMMARY OF THE INVENTION

To address the above-discussed deficiencies of the prior art, the present invention provides, in one aspect, a suction catheter. In one embodiment, the suction catheter includes: (1) a housing portion having an interior passage therethrough, the interior passage couplable to a vacuum source, (2) a tube portion coupled to the housing portion protruding from the housing a length based on a length of an endotube to be suctioned and (3) a bypass valve associated with the housing portion and configured to control suction through the tube portion.

In another aspect, the present invention provides a method of manufacturing a suction catheter. In one embodiment, the method includes: (1) forming a housing portion having an interior passage therethrough and a bypass valve configured to control suction through the tube portion and (2) coupling a tube portion to the housing portion, the tube portion protruding from the housing a length based on a length of an endotube to be suctioned.

The foregoing has outlined preferred and alternative features of the present invention so that those skilled in the pertinent art may better understand the detailed description of the invention that follows. Additional features of the invention will be described hereinafter that form the subject of the claims of the invention. Those skilled in the pertinent art should appreciate that they can readily use the disclosed conception and specific embodiment as a basis for designing or modifying other structures for carrying out the same purposes of the present invention. Those skilled in the pertinent art should also realize that such equivalent constructions do not depart from the spirit and scope of the invention.

### BRIEF DESCRIPTION OF THE DRAWINGS

For a more complete understanding of the invention, reference is now made to the following descriptions taken in conjunction with the accompanying drawing, in which:

FIG. 1-A is a side view of one embodiment of a suction catheter constructed according to the principles of the present invention;

FIG. 1-B is a top view of the embodiment of FIG. 1-A;

FIG. 2 is a side view of the embodiment of FIG. 1-A and includes a side view of a protective sheath slipped over both the suction catheter device and the rubber flaring of the suction hose;

FIG. 3-A is a side view of an alternative embodiment of a suction catheter constructed according to the principles of the present invention that includes a hollow, cylindrical spacer incised with helical threads to set the length of the elongated tubing available to enter the tracheal tube;

FIG. 3-B is a side view of an alternative embodiment of a suction catheter constructed according to the principles of the present invention that includes a family of nested, fixed-length spacers to set the length of the elongated tubing;

FIG. 3-C is a side view of an alternative embodiment of a suction catheter constructed according to the principles of the present invention that includes a threaded spacer attached to the elongated tubing that seats into the male coupling to set the length of the elongated tubing;

FIG. 4-A is a side view of an alternative embodiment of a suction catheter constructed according to the principles of the present invention that includes a wire embedded into the elongated tube to provide lateral strength;

FIG. 4-B is a side view of an alternative embodiment of a suction catheter constructed according to the principles of the present invention that includes a telescoping sheath to protect and guide the elongated tube into the tracheal tube;

FIG. 4-C is a side view of an alternative embodiment of a suction catheter constructed according to the principles of the present invention that includes a corrugated sheath to protect and guide the elongated tube into the tracheal tube;

FIG. 5 schematically illustrates a method of removing an obstruction from an endotube using the embodiment of the suction catheter illustrated in FIG. 1-B with the catheter held between the thumb and index finger to show the grip for single-hand operation;

FIG. 6-A schematically illustrates an embodiment of a suction catheter that does not extend beyond the end of the tracheal tube when the catheter is fully inserted;

FIG. 6-B schematically illustrates a prior art suction catheter that is capable of reaching to the carina where it may cause damage to surrounding mucosa; and

FIG. 7 is a flow diagram of one embodiment of a method of manufacturing a suction catheter carried out according to the principles of the present invention.

### DETAILED DESCRIPTION

In certain embodiments, the present invention provides a tracheal tube length suction catheter that can be used to quickly and easily aspirate secretions from tracheostomy and endotracheal tubes. After each use the catheter can be safely stored in a clean encasement that slips over the catheter and grips to the outside of the connection tubing.

While animal studies clearly demonstrate denuded epithelium and inflammation where suctioning past the tracheal tube is routinely performed, the majority of clinicians often still deep-suction their patients. Abandoning the practice has been advocated for more than a decade, though with little change in routine. By providing a length-adjustable catheter or sizing a family of catheters to match the length of tracheal tubes, as described herein, the clinician is physically unable to suction past the end of the tracheal tube and injury to the airway will be prevented.

In certain embodiments of the present invention, the suction catheter's length is equivalent to the length of a tracheal tube. Thus, the catheter tubing for most tracheostomy tubes will not extend beyond a couple of centimeters and therefore support its own weight laterally without sagging. With this short assembly the clinician is able to insert the catheter into a tracheal tube with a motion similar to that of a driver inserting a key into an ignition. Because the catheter housing is wider than the opening of the tracheal tube, penetration will stop when the housing meets the connector of the tracheal tube.

The clinician is able to quickly and easily insert the catheter into the tracheostomy tube with only a single hand. She can then suction secretions effectively by swirling the tip of the catheter with her wrist while controlling the vacuum pressure with the bypass valve by using either a thumb or finger on the same hand. Grasping the catheter housing with the thumb and index finger and immediately thrusting the catheter into the tracheal tube is much more time efficient than sliding back a catheter sleeve, finding the pre-measured depth mark and then inserting with vigilant care.

A single length-adjustable catheter may also be used instead of a family of catheters to match the lengths of tracheal tubes. Certain embodiments of the length-adjustable catheter may use a spacer that seats into the central housing body or a clamp located inside to the central housing body that allows the elongated catheter tubing to be slid into position and then clamped to match the length of the tracheal tube.

A spacer that seats into the front of the central housing body has an edge diameter wider than the opening of the tracheal tube to stop penetration when it meets the tracheal tube connector. Spacing may be accomplished with a single cylindrical rod incised with one or more helical or advancing spiral threads. The rod is hollow to allow the elongated catheter tubing to pass through. As the rod is rotated in one direction, it seats deeper into the central housing body, thereby increasing the effective length of the elongated tubing available to enter the tracheal tube. If it were rotated in the other direction, it decreases the effective length of the elongated catheter tubing. Spacing could also be accomplished by using a family of nested, fixed-length buttons also hollowed to allow the catheter tubing to pass through. As many spacers as needed may be used to set the effective length of the elongated catheter tubing.

In certain embodiments of the present invention, a spacer that seats into the male coupling in back of the central housing body is permanently attached to the elongated catheter tubing. The spacer might also be threaded. As the spacer is rotated in one direction, the spacer seats into the central housing body and pushes the elongated tubing through to increase the effective length available to enter the tracheal tube. Rotating in the opposite direction has the opposite effect. In certain embodiments, it is necessary that the spacer's diameter be smaller than the male coupling inlet. This allows air from the aperture to bypass the elongated catheter tubing and keeps negative pressure from building at the tip of the catheter until demanded by a user covering the aperture. A spacer might be avoided if the elongated tubing is allowed to slide to a desired length, at which point an externally controlled clamp could hold the elongated tubing in position inside the central housing body.

Tracheostomy tube lengths typically vary between 2 cm and 10 cm, but endotracheal tubes can be much longer, up to and over 15 cm in length. Because requirements on the flexibility of the tracheal tube can be different across patients, a need may arise for the catheter to stay extremely pliable, even at greater lengths. If these highly pliable catheters extend beyond a certain length an alternative guidance system may be needed.

Alternative embodiments may employ either a telescoping or a corrugated sheath to maintain the lateral posture of the catheter tubing until it can reach the tracheal tube connector. As the catheter housing is pushed in toward the patient, the sheath catches on the connector and compresses back toward the clinician while the catheter tubing continues on, sliding into the tracheal tube. The tubes align at the point at which the sheath has no more room to compress. If less flexibility is required, a guide wire may be embedded in the catheter tubing to maintain enough lateral rigidity to prevent drooping but preserve enough flexibility to conform to the curve of the tracheal tube.

Catheters used in a home environment are difficult to keep clean because they are used over and over throughout the day. They are stored in proximity to their vacuum source and are often placed under a pillow, draped over a crib railing, slipped into an adjacent drawer or tucked into the suction machine bag where they might fall to the floor. Mucus can mix with outside contaminates, spread up the entire length of the catheter and deposit on both the inside and the outside of the protective sleeve.

Certain embodiments of the present invention therefore include a semi-rigid sheath that couples to the outside of the rubber flaring of the source vacuum tube to encase the entire suction catheter. The sheath provides clean storage of the catheter in a variety of environments and allows the catheter to be used repeatedly over a 24-hour period at which point the catheter may be discarded. When a clinician wants to use the catheter, she grabs the outside of the sheath and pulls it off of the flaring of the suction hose. The uncontaminated catheter is immediately ready to for use.

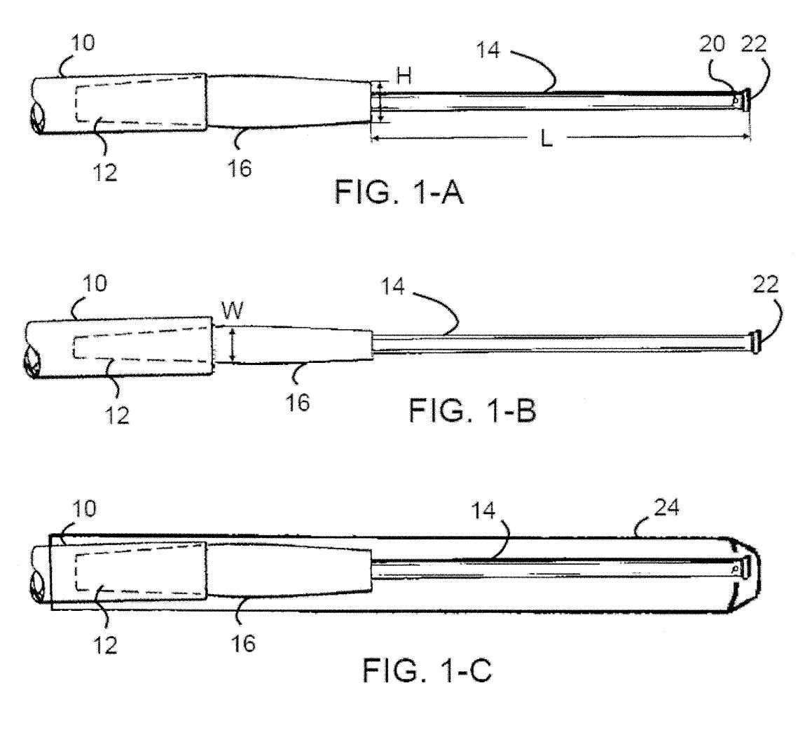

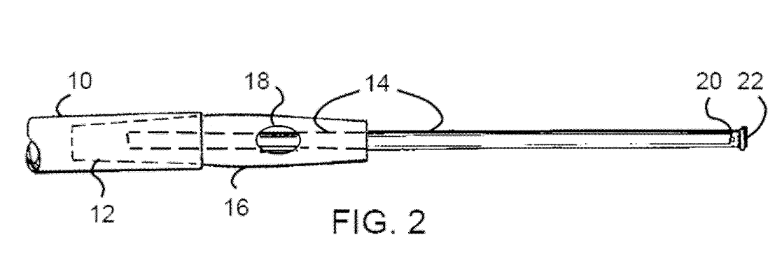

One embodiment of the present invention will now be illustrated and described. A suction catheter assembly with rigid protective sheath 24, is illustrated in FIG. 1-A (side view), FIG. 1-B (top view) and FIG. 2. A suction catheter includes a central housing body 16 with a bypass valve aperture 18 (FIG. 2), a suction hose male coupling 12 and elongated catheter tubing 14 with side inlets 20 and a tip inlet 22.

A variety of known materials may be used to manufacture these catheter assemblies. The suction catheters defined herein can be made of relatively flexible plastic material such as natural or synthetic rubber, polypropylene, polyethylene, polyvinyl chloride, nylon or like material having the flexibility and resilience necessary to suction the tracheobronchial secretions from the airway of an intubated patient.

At one end of the suction catheter, the suction hose male coupling 12 plugs into a rubber flaring 10 of a suction hose. The suction hose is connected on the other side to a secretion collection canister to which negative pressure can be applied by a conventional vacuum source (not shown). The suction hose male coupling 12 is made of rigid plastic and tapers forward into the central housing body 16 to form a continuous rigid frame onto which a clinician can hold while using the suction catheter.

The elongated catheter tubing 14 protrudes forward from the central housing body 16 for a length L, which varies from catheter to catheter but which approximately matches (within a centimeter in some embodiments) the length of a given tracheal tube 42. Near the distal end of the elongated catheter tubing 14 are side inlets 20 and at the very end a tip inlet 22 that provide the ports into which secretions are vacuumed. The elongated catheter tubing 14 also recedes back in through the central housing body 16 and into the suction hose male coupling 12. Because the end of the elongated catheter tubing 14 lies past the bypass valve aperture 18, the operator's control finger remains isolated from the suction fluid stream while gripping the central housing body 16.

The bypass valve aperture 18 is provided to regulate flow at the distal inlets 20, 22. When the bypass valve aperture 18 is left uncovered and open, air is allowed to enter from that aperture to prevent any significant pressure at the tip. When the valve is closed, air and respiratory secretions are drawn in through the inlets 20, 22 of the catheter.

The illustrated embodiment of the bypass valve aperture 18 is a simple cutout from the side of the central housing body 16. The size and shape of the bypass valve aperture 18 ensures a tight seal when covered with the tip of a finger, thus enabling negative pressure control by the clinician. The side position of the bypass valve aperture 18 and the narrow width W of the central housing body 16 provide an opportunity for the clinician to grip the assembly with the pinch of a finger and thumb. This hand positioning, versus a thumb forward position, keeps the clinician from interfering with the pre-measured length L of the elongated catheter tubing 14 as it is inserted into a tracheal tube 42.

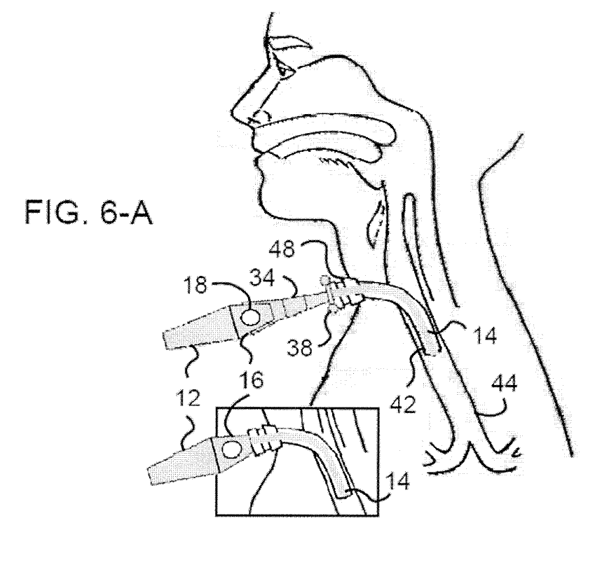

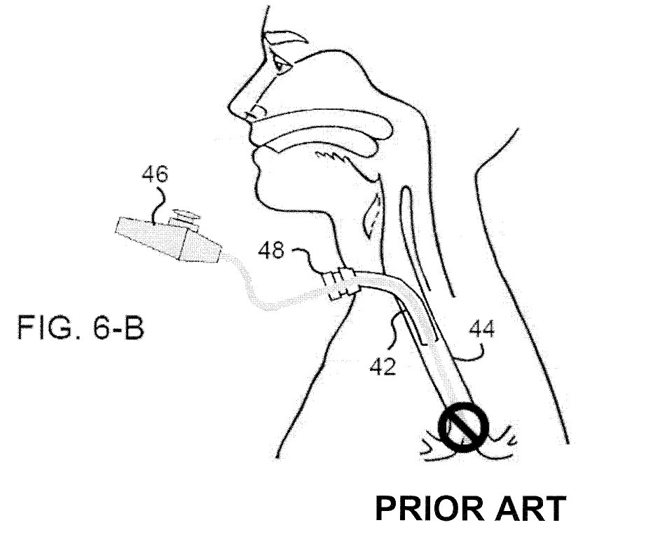

Because the hand is positioned behind the central housing body 16, the clinician can insert the catheter to its full length L until the most distal portion of the central housing body 16 with height H reaches the tracheal tube connector 48 (FIG. 6-A). Because the height H of the central housing body 16 is greater than the inner diameter of the tracheal tube connector 48, the central housing body 16 halts on the connector and proceeds no farther. In one embodiment, the elongated catheter tubing 14 cannot extend past the point at which its end aligns with the end of the tracheal tube 42. In alternative embodiments, the elongated catheter tubing stops just short of, or extends slightly past, the end of the tracheal tube 42.

A combination of the following may be particularly advantageous: (i) an elongated catheter tubing 14 substantially equal to the length of the tracheal tube 42, (ii) an elongated catheter tubing 14 that recedes into the suction hose male coupling 12 and (iii) and a central housing body 16 with a bypass valve aperture 18 faced generally orthogonally. This combination allows the operator the maximum possible dexterity when using the catheter.

FIG. 2 illustrates the embodiment of the suction catheter assembly of FIG. 1-A sealed within a rigid protective sheath 24, which provides clean storage of the catheter in a variety of environments and allows the catheter to be used repeatedly over an entire day. The rigid protective sheath 24 is, at a minimum, as long as the entire suction catheter and couples to the outside of the suction hose rubber flaring 10 to encase the entire suction catheter. When a clinician wants to use the catheter, she can grasp the outside of the rigid protective sheath 24 and simply pull it off the rubber flaring 10. The assembly is always in a state of readiness without compromising the cleanliness of the central housing body 16 or more importantly the elongated catheter tubing 14. The rigid protective sheath 24 keeps the catheter assembly clean and prevents mucous, which might remain on the outside of the elongated catheter tubing 14 after being used by a clinician, from mixing with foreign contaminates and spreading up the entire length of the elongated catheter tubing 14.

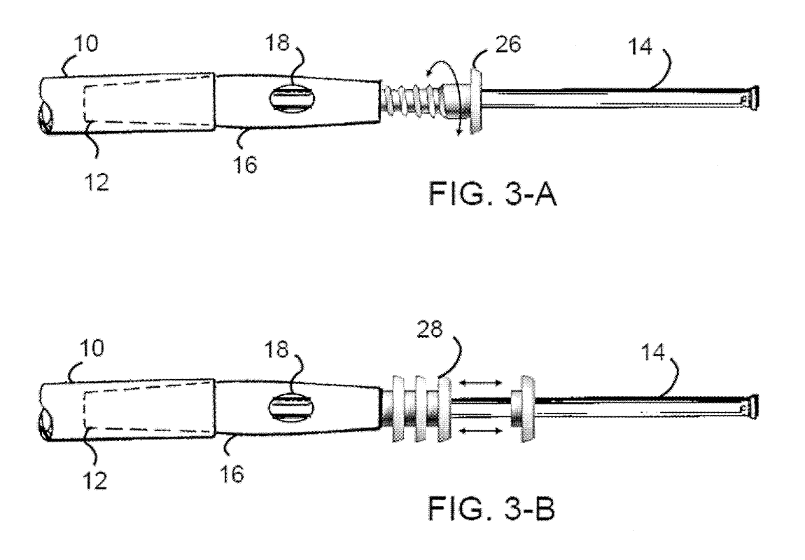

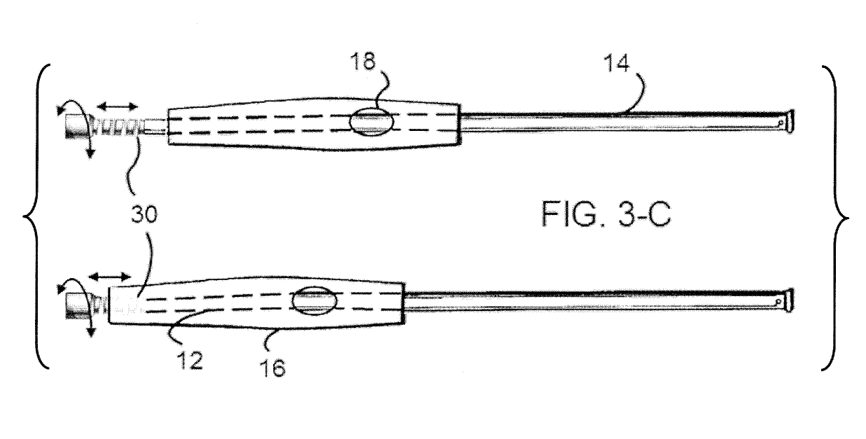

Alternative embodiments of the present invention, as illustrated in FIG. 3-A, FIG. 3-B and FIG. 3-C, are constructed in a manner similar to that of the primary embodiment with a central housing body 16, a bypass valve aperture 18, a suction hose male coupling 12 and elongated catheter tubing 14. All of the embodiments in FIG. 3 provide options for a length-adjustable catheter by using a spacer that seats into the central housing body 16. Each option provides a mechanism by which the clinician can set the length of the elongated catheter tubing 14 to match the length of a to-be suctioned tracheal tube 42.

Referring to FIG. 3-A, this particular embodiment uses a threaded spacer 26 that seats into the front of the central housing body 16. Because the diameter of the threaded spacer 26 is greater than the inner diameter of the tracheal tube connector 48, it catches on the connector and prevents the elongated catheter tubing 14 from sinking deeper into the tracheal tube 42. The threaded spacer 26 is a cylindrical rod incised with advancing helical spiral threads and is hollowed to allow the elongated catheter tubing 14 to pass through. As the threaded spacer 26 is rotated in one direction, it seats deeper into the central housing 16, thereby increasing the effective length of the elongated catheter tubing 14 available to travel the length of the tracheal tube 42. If the threaded spacer 26 is rotated in the other direction it decreases the effective length of the elongated catheter tubing 14.

Referring to FIG. 3-B, this particular embodiment uses a series of nested spacers 28, which position in front of the central housing body 16. The nested spacers 28 are hollowed to allow the elongated catheter tubing 14 to pass through. The diameter of forward edge of the nested spacer 28 is greater than the inner diameter of the tracheal tube connector 48. Nested spacers 28 are added, each with a known width, to set the effective length of the elongated catheter tubing 14 so that it cannot extend past the end of the tracheal tube 42.

Referring to FIG. 3-C, this particular embodiment employs a tubing-connected spacer 30, which seats into suction hose male coupling 12 in back of the central housing body 16. The tubing-connected spacer 30 is permanently fixed by the manufacturer to the elongated catheter tubing 14. The tubing-connected spacer 30 is threaded so as it is rotated in one direction, it seats deeper into the central housing body 16 to push the elongated catheter tubing 14 and increase the length available to enter the tracheal tube 42. If the tubing-connected spacer 30 is rotated in the other direction it decreases the effective length of the elongated catheter tubing 14. It is desirable for the tubing-connected spacer's 30 diameter to be smaller than the male coupling inlet. This allows air from the bypass valve aperture 18 to pass to the suction device when left uncovered and keep pressure from building at the inlets 20, 22 of the elongated catheter tubing 14. A tubing-connected spacer 30 might also be effected by allowing the elongated catheter tubing 14 to slide to a desired length and then an externally controlled clamp could hold it in position inside the central housing body 16.

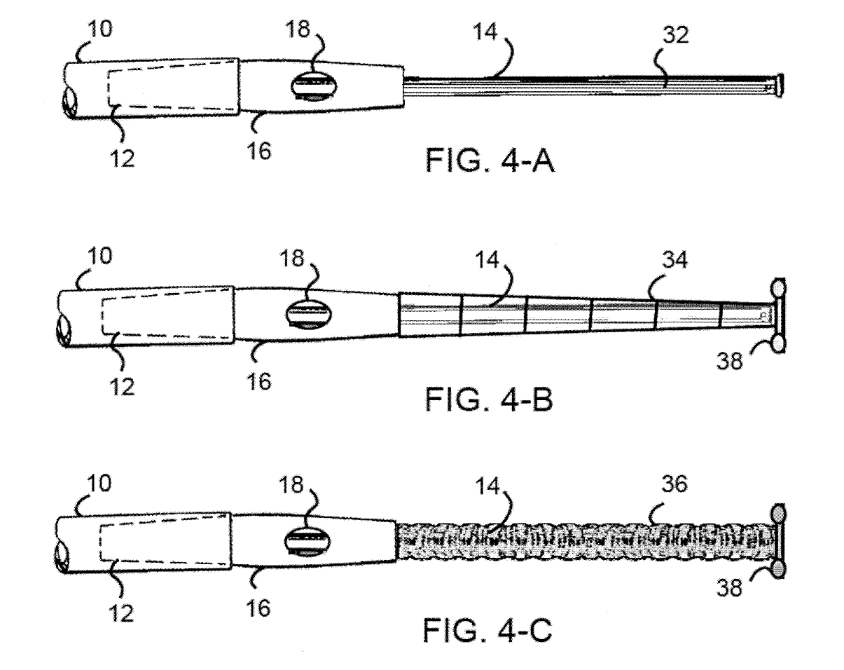

Alternative embodiments, as illustrated in FIG. 4-A, FIG. 4-B and FIG. 4-C, may be constructed in a manner similar to that of the primary embodiment with a central housing body 16, a bypass valve aperture 18, a suction hose male coupling 12 and elongated catheter tubing 14. All of the embodiments of FIG. 4 provide mechanisms to help the clinician guide the elongated catheter tubing 14 into the tracheal tube connector 48 (FIG. 6-A). They also provide extra protection for the elongated catheter tubing 14 while the suction catheter is dislodged from the rigid protective sheath 24.

Referring to FIG. 4-A, this particular embodiment embeds a guide wire 32 into the elongated catheter tubing 14. The embedded guide wire 32 might be used in suction catheters with longer catheter tubing 14 to maintain enough lateral rigidity to prevent drooping but still preserve enough flexibility to conform to the curve of the tracheal tube 42.

Referring to FIG. 4-B, this particular embodiment employs a telescoping sheath 34 to maintain lateral rigidity until the elongated catheter tubing 14 can reach the tracheal tube connector 48. As the central housing body 16 is pushed in toward the patient, the sheath guard 38 catches on the tracheal tube connector 48 and compresses back toward the clinician while the elongated catheter tubing 14 continues on, sliding into the tracheal tube 42. The tubes align at the point at which the telescoping sheath 34 has no more room left to compress.

Referring to FIG. 4-C, this particular embodiment employs a corrugated sheath 36 in a manner similar to the telescoping sheath 34 to maintain lateral rigidity of the elongated catheter tubing 14 as it is guided into the tracheal tube connector 48. It also compresses in a predefined manner after the sheath guard 38 catches on the tracheal tube connector 48 until the point at which the tubes align.

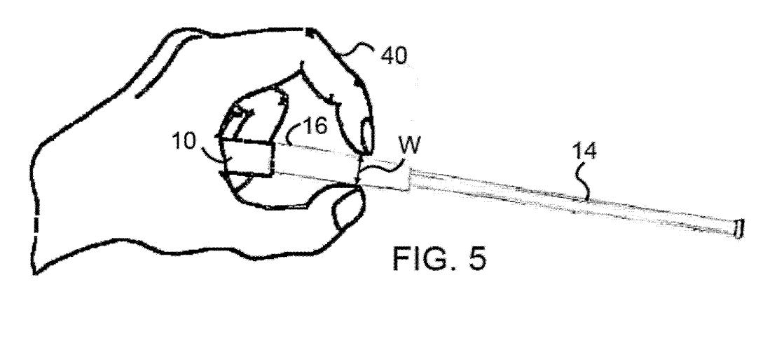

Operation of the embodiment of FIG. 1-A is illustrated in FIG. 5, and the operation of one alternative embodiment is illustrated FIG. 6-A, though each embodiment could be interchangeably illustrated in either FIGURE.

Referring to FIG. 5, the diagram shows the embodiment of FIG. 1-A, as a human hand 40 might grip it. The thumb and index finger grip the sides of the central housing body 16 and either digit can be used to cover the bypass valve aperture 18. Part of the suction hose and rubber flaring 10 connected to the suction catheter are gripped in the palm of the hand 40 as the catheter is used. FIG. 5 illustrates the relatively short length L of the elongated catheter tubing 14 and demonstrates how easy it is to control the assembly while inserting the catheter or while suctioning a patient.

FIG. 6-A and FIG. 6-B compare and contrast the utilization of an alternative embodiment of the invention with that of a commonly used suction catheter 46. FIG. 6-B shows how such a commonly used suction catheter 46 may be misused while suctioning a patient with a tracheostomy tube. Illustrated is the entry of the known suction catheter into the trachea 44 of the patient all the way to the carina. This form of deep-suctioning can cause tissue damage due to the inflow of mucous membrane into the apertures on the distal tip of the catheter. Conversely, FIG. 6-A shows how the use of the alternative embodiment of FIG. 4-B can effectively protect the lower trachea 44 from damage. FIG. 6-A demonstrates the telescoping sheath 34 already fully compressed against the tracheal tube connector 48 with the elongated catheter tubing 14 aligned with the end of the tracheal tube 42.

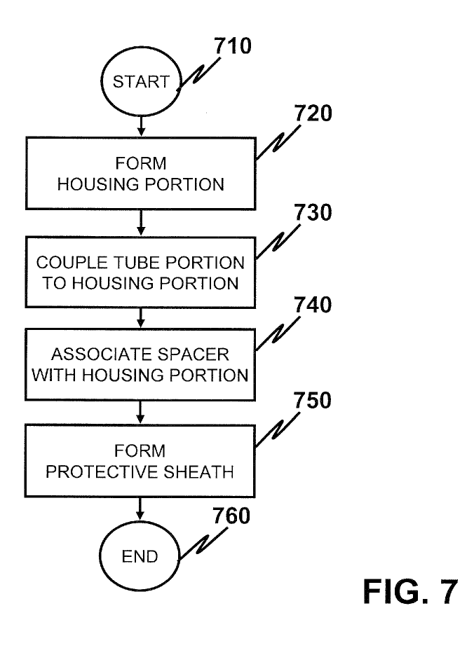

Turning now to FIG. 7, illustrated is a flow diagram of one embodiment of a method of manufacturing a suction catheter carried out according to the principles of the present invention. The method begins in a start step 710.

In a step 720, a housing portion is formed. The housing portion has an interior passage therethrough and a bypass valve configured to control suction through the tube portion.

In a step 730, a tube portion is coupled to the housing portion. The tube portion protrudes from the housing a length based on a length of an endotube to be suctioned. The tube portion includes a tip inlet and at least one side inlet.

In a step 740, a spacer is associated with the housing portion. The spacer has a height greater than an inner diameter of a connector for the endotube. The spacer may be translatable with respect to the housing portion and perhaps threaded to allow the spacer to be translatable with respect to the housing portion. At least an additional stackable spacer may be associated with the spacer as described above.

In a step 750, a protective sheath is formed. The protective sheath has a length based on the length of the tube portion. The protective sheath may be wholly removable from the suction catheter or configured to retract toward the housing portion to expose the tube portion.

The method ends in an end step 760. The various steps of the method may be carried out in any advantageous order. Various steps of the method may be omitted or augmented as may be advantageous to a particular application.

Although the present invention has been described in detail, those skilled in the pertinent art should understand that they can make various changes, substitutions and alterations herein without departing from the spirit and scope of the invention in its broadest form.

What is claimed is:

1. A suction catheter, comprising:
a housing portion having an interior passage therethrough, said interior passage couplable to a vacuum source; and
a tube portion coupled to said housing portion protruding from said housing a length based on a length of an endotube to be suctioned.

2. The suction catheter as recited in claim 1 wherein said endotube is selected from the group consisting of:
an endotracheal tube, and
a tracheostomy tube.

3. The suction catheter as recited in claim 1 further comprising a bypass valve associated with said housing portion and configured to control suction through said tube portion.

4. The suction catheter as recited in claim 1 wherein said housing has a height greater than an inner diameter of a connector for said endotube.

5. The suction catheter as recited in claim 1 further comprising a spacer associated with said housing portion and having a height greater than an inner diameter of a connector for said endotube.

6. The suction catheter as recited in claim 5 wherein said spacer is translatable with respect to said housing portion.

7. The suction catheter as recited in claim 5 further comprising at least an additional stackable spacer.

8. The suction catheter as recited in claim 1 further comprising a protective sheath coupled to said housing portion and configured to retract toward said housing portion to expose said tube portion.

9. The suction catheter as recited in claim 1 wherein said housing portion comprises a male connector.

10. The suction catheter as recited in claim 1 wherein an outer diameter of said tube portion is approximately equal to an inner diameter of said endotube.

11. The suction catheter as recited in claim 1 wherein said tube portion further comprises a tip inlet and at least one side inlet.

12. A protective sheath, comprising:
a body having an opening at a proximal end thereof, a closure at a distal end thereof, and an interior passage therethrough, said body having a length approximately equal to a suction catheter.

13. The protective sheath as recited in claim 12 wherein said proximal end couples to a connector of an external vacuum source, said protective sheath being of sufficient width to slip over said suction catheter and form a substantial seal with said external vacuum source connector.

14. The protective sheath as recited in claim 12 wherein said proximal end couples to said suction catheter with said protective sheath being of sufficient width to slip over said suction catheter and form a substantial seal with a body portion of said suction catheter.

15. A method of manufacturing a suction catheter, comprising:
forming a housing portion having an interior passage therethrough; and
coupling a tube portion to said housing portion, said tube portion protruding from said housing a length based on a length of an endotube to be suctioned.

16. The method as recited in claim 15 wherein said endotube is selected from the group consisting of:
an endotracheal tube, and
a tracheostomy tube.

17. The method as recited in claim 15 wherein said housing portion has a bypass valve configured to control suction through said tube portion.

18. The method as recited in claim 15 wherein said housing has a height greater than an inner diameter of a connector for said endotube.

19. The method as recited in claim 15 further comprising associating a spacer with said housing portion, said spacer having a height greater than an inner diameter of a connector for said endotube.

20. The method as recited in claim 19 wherein said spacer is translatable with respect to said housing portion.

21. The method as recited in claim 19 further comprising associating at least an additional stackable spacer with said spacer.

22. The method as recited in claim 15 further comprising a forming a protective sheath having a length based on said length of said tube portion.

23. The method as recited in claim 22 wherein said protective sheath is configured to retract toward said housing portion to expose said tube portion.

24. The method as recited in claim 15 wherein said housing portion comprises a male connector.

25. The method as recited in claim 15 wherein an outer diameter of said tube portion is approximately equal to an inner diameter of said endotube.

26. The method as recited in claim 15 wherein said tube portion further comprises a tip inlet and at least one side inlet.

27. A method of removing an obstruction from an endotube, comprising:
coupling a suction catheter to an external vacuum source;
grasping said suction catheter using a single hand;
positioning a distal end of said suction catheter into an opening of said endotube without requiring a second hand;
passing said suction catheter through said endotube to remove said obstruction; and
retracting said suction catheter from said endotube, a length of said suction catheter preventing said user from inserting said distal portion substantially deeper than a distal portion of said endotube.
